(19) United States
(12) Patent Application Publication
(10) Pub. No.: US 2018/0192944 A1
(43) Pub. Date: Jul. 12, 2018

(54) METHODS FOR MONITORING COMPOSITIONAL CHANGES IN A BODY

(71) Applicant: KLARISMO, INC., San Francisco, CA (US)

(72) Inventors: Ignatius Dewet Diener, London (GB); Marcus Foster, San Francisco, CA (US); David Greer, London (GB); Kevin Keraudren, London (GB); Brandon Whitcher, London (GB)

(21) Appl. No.: 15/419,983

(22) Filed: Jan. 30, 2017

Related U.S. Application Data
(63) Continuation of application No. PCT/US2017/012891, filed on Jan. 10, 2017.
(60) Provisional application No. 62/450,037, filed on Jan. 24, 2017.

(51) Int. Cl. A61B 5/00; G06T 7/00; G06T 7/62; A61B 5/055

(52) U.S. Cl. CPC A61B 5/4872; G06T 7/0016; G06T 7/62; G06T 2207/30004; G06T 2207/10088; G06T 2207/30196; A61B 5/055

(57) ABSTRACT

Within this disclosure are new methods of monitoring compositional changes of a body. In one embodiment, the methods comprise three-dimensional models of a body. In one embodiment, two-dimensional MRI images are used to create a three-dimensional model of a body. In one embodiment, the three-dimensional model is segmented and landmarked to provide reference points for monitoring changes. In some embodiments, volumetric values are calculated to provide data about changes the body has undergone. In one embodiment, the body is a human body.

### CROSS REFERENCE TO RELATED APPLICATIONS

This application is a Continuation of International Application Serial Number PCT/US17/12891, entitled “METHODS FOR PROCESSING THREE DIMENSIONAL BODY IMAGES,” filed Jan. 10, 2017 and claims benefit of Provisional Application Ser. No. 62/450,037, entitled “METHODS FOR MONITORING COMPOSITIONAL CHANGES IN A BODY,” filed Jan. 24, 2017, which are hereby incorporated by reference in their entirety.

### TECHNICAL FIELD

This disclosure relates to body imaging and analysis. In particular, this disclosure relates to tracking the changes of a body over time.

### BACKGROUND

Diet and exercise are important for maintaining personal health and fitness. Many different diets and exercise trends have come and gone, all promising to lower body weight. However, body weight is not the only factor in maintaining personal health.

While many strive for good health they do not fully comprehend how important it is to learn about the interior of their bodies. People focus on how their body looks from the outside but rarely think about the condition of the interior of their body. Understanding the internal conditions of one's body is possibly the most important factor in maintaining proper health. As such, having internal images of a body is important to properly evaluate one's health.

Common medical imaging techniques include X-ray, Magnetic Resonance Imaging (MRI), and Computerized Tomography (CT) scans. One advantage MRI scans have over CT scans is that MRI does not expose the patient to ionizing radiation that could cause potential side effects. MRI scans provide a high level of detail, resolution, and clarity of the anatomy and physiology of the human body.

MRI scans are traditionally done because there is a specific need to investigate, e.g., cancer concerns, diseases, organ issues, etc. MRI scans are not traditionally used for a routine examination. As such, an MRI scan only provides one glimpse of the body at a time. Even with an MRI scan, most medical professionals focus on a small specific part of the body. At present, much of the information contained within an MRI scan is left unused. Another limitation of the current state of the art is that radiologists perform qualitative assessments of body images whereas analyzing the images quantitatively would provide for improvements in speed, efficiency, accuracy, etc.

Most patients are not capable of using an MRI image for their own personal benefit. Patients will even sometimes receive a disk of their MRI images but not have the ability and tools to fully extrapolate the information they have about their body. An MRI scan can provide incredibly useful information for about one's entire body and could be used to develop diets, exercise plans, treatments, etc.

There exists a need for using internal images of the body for personal health. There also exists the need for three-dimensional representations of the body representing changes to the body. There also exists a need to monitor changes of a body over time. There exists a need for calculating volumetric measurements of the body for quantifying changes to the body. There also exists a need for automating images presenting steps to create volumetric comparisons of a body. There exists a need for a graphical user interface for presenting graphical representations of a body showing changes. There exists a need for using MRI scans for producing data of changes to the body. There also exists a need for segmenting images of a body for monitoring changes to the body.

### DETAILED DESCRIPTION

Disclosed herein is a new method for measuring compositional changes in a body. In one embodiment, compositional changes are measured between two time periods. In one embodiment, compositional changes are measured between multiple time periods. In one embodiment, compositional changes are presented on a graphical representation.

Disclosed herein is a new representation of a body for monitoring changes. In one embodiment, the body is a human. In one embodiment, the body is a three-dimensional representation. In one embodiment, the representation of the body illustrating differences in one representation of the body compared to another representation of the body.

Disclosed herein is a new method of automating internal images of a body for monitoring changes. In one embodiment, automating comprises a cloud database platform for storing changes over time. In one embodiment, automating comprises a machine learning program for identifying changes over time. In one embodiment, automating comprises updating representations of the body with new internal images.

Disclosed herein is a new method of utilizing MRI scans. In one embodiment, MRI scans are compiled for creating a three-dimensional representation of the body. In one embodiment, MRI scans are used for displaying changes to the body. In one embodiment, MRI scans are used to calculate volumetric measurements to illustrate changes in the body.

Disclosed herein is a new graphical user interface. In one embodiment, the graphical user interface marks changes in a body. In one embodiment, the graphical user interface displays a plurality of images to track changes. In one embodiment, the graphical user interface allows a user to highlight changes. In one embodiment, the graphical user interface allows a user to control a representation of a body. In one embodiment, the user can select segments. In one embodiment, the user can select different points in time. In one embodiment, the user can monitor changes over time. In one embodiment, the user selects different points in time for comparing representations of the body. In one embodiment, the user overlaps representations of the body.

Disclosed herein is a method of measuring compositional change in a body, comprising:
Collecting a first plurality of two-dimensional images for a body at a first time;
Collecting a second plurality of two-dimensional images for a body at a second time;
Processing the first plurality of two-dimensional body images into a first three-dimensional representation of the body;
Processing the second plurality of two-dimensional body images into a second three-dimensional representation of the body;
Assigning landmarks to points within the first three-dimensional representation of the body;
Assigning landmarks to points within the second three-dimensional representation of the body;
Registering the first three-dimensional representation of the body;
Registering the second three-dimensional representation of the body;
Segmenting the first three-dimensional representation of the body into a first segment;
Segmenting the second three-dimensional representation of the body into a second segment;
Calculating a volume of the first segment; and
Calculating a volume of the second segment.

As used herein, the term “collecting” means gathering, receiving, transferring, downloading, and/or finding. In one embodiment, collecting comprises receiving information from a cloud database. In one embodiment, collecting comprises transferring information from a disk onto a computer. In one embodiment, collecting comprises transferring information from a USB drive onto a computer. In one embodiment, collecting comprises streaming. In one embodiment, collecting comprises receiving information from different points in time. In one embodiment, collecting is gathering data for calculating differences.

In one embodiment, collecting comprises receiving images from an MRI machine. An MRI machine changes the orientation of protons in the body using magnetic energy. As the protons return to their original orientation, they produce radio signals which are recorded. Protons behave differently in different tissues of the body producing different radio signals allowing an MRI machine to differentiate between the different tissues of the body. The intensity of the received signal is then plotted on a grey scale and cross sectional images are built. In one embodiment, the MRI machine stores images in DICOM format. In one embodiment, collecting comprises receiving multiple images from multiple MRI machines. In one embodiment, collecting comprises receiving multiple images from multiple MRI machines from multiple points in time.

Within the context of this disclosure, collecting encompasses passively receiving information in which information is collected without request. Within the context of this disclosure, collecting encompasses actively receiving information in which information is requested.

As used herein, the term “plurality” refers to more than one. In one embodiment, there is a plurality of images for one body. In one embodiment, there is a plurality of two-dimensional images of the body. In one embodiment, there is a plurality of planar slices of the body. In one embodiment, there is a plurality of parts of the same body. In one embodiment, there is a plurality of images for a plurality of bodies. In one embodiment, there is a plurality of images for one section of a body. In one embodiment, there is a plurality of bodies. In one embodiment, there is a plurality of changes.

As used herein, the term “two-dimensional” refers to an object or image appearing to have width and length, i.e., area, within a plane. Area is the quantity that expresses the extent of a two-dimensional figure or shape in the plane. In one embodiment, two-dimensional comprises Cartesian coordinates. In one embodiment, an image is in the x and y axis. In one embodiment, a body is in the x and z axis. In one embodiment, a body is in the y and z axis. In one embodiment, two dimensional comprises polar coordinates.

As used herein, the term “image” refers to a likeness or representation of a person, animal, or thing, photographed, painted, sculptured, and/or otherwise made visible. In one embodiment, an image is a digital image. In one embodiment, an image is a picture of a human body. In one embodiment, an image is from an MRI machine. In one embodiment, an image is a collection of pixels. In one embodiment, the image is in a JPEG format. In one embodiment, the image is in DICOM format. In one embodiment, the image is in NIfTI format. In one embodiment, the image illustrates a change in a body.

As used herein, the term “two-dimensional image” refers to a presentation of mass present within a particular plane, e.g., cross-section of a body. In one embodiment, the two-dimensional image comprises Cartesian coordinates, e.g., x, y, and z axis. In one embodiment, the two-dimensional image is a picture. In one embodiment, the two-dimensional image is a digital image on a screen. In one embodiment, there are two-dimensional images from two time periods.

As used herein, the term “body” refers to the physical structure and the material substance of a mass, e.g., an organism. In one embodiment, the body is living. In one embodiment, the body is not living. In one embodiment, the body is a human. In one embodiment, the body is a dog. In one embodiment, the body is a horse. In one embodiment, the body comprises organs. In one embodiment, the body comprises a skeleton. In one embodiment, the body comprises fat. In one embodiment, the body comprises muscles. In one embodiment, the body comprises subcutaneous fat. In one embodiment, the body comprises adipose tissue. In one embodiment, the body comprises visceral fat. In one embodiment, the body comprises iliopsoas muscles.

As used herein, the term “image of a body” refers to a representation of a physical structure and material substance of a physical mass, e.g., an organism. In one embodiment, the image of a body is an embodiment of an MRI scan. In one embodiment, the image of a body is an embodiment of a CT scan. In one embodiment, the image of a body is an internal image. In one embodiment, the image of a body is of a human body or a portion of a human body.

As used herein, the term “processing” refers to treating a thing through a series of steps. In one embodiment, processing comprises using a computer algorithm. In one embodiment, processing comprises producing an image from another image. In one embodiment, processing comprises using a processor. In one embodiment, processing comprises manually manipulating an image. In one embodiment, processing comprises creating a three-dimensional image from a series of two-dimensional crosscut images. In one embodiment, processing comprises analyzing segments of a body. In one embodiment, processing comprises monitoring changes of a body.

As used herein, the term “three-dimensional” refers to an object or image appearing to have length, depth, and breadth, i.e., volume. Volume is the amount of space occupied by an object. In one embodiment, volume is measured in cubic meters (m3). In one embodiment, three dimensional comprises Cartesian coordinates. In one embodiment, an image has multiple x, y, and z coordinates. In one embodiment, three-dimensional illustrates changes to a body.

As used herein, the term “representation of the body” refers to a concrete portrayal of a physical structure having material substance. In one embodiment, the representation of the body is two-dimensional. In one embodiment, the representation of the body is three-dimensional. In one embodiment, the representation of the body is a representation of a human body. In one embodiment, the representation of the body is a picture. In one embodiment, the representation of the body is a digital image. In one embodiment, the representation of the body is a representation of the skeleton. In one embodiment, the representation of the body is an MRI scan. In one embodiment, the representation of the body is a CT scan. In one embodiment, the representation of the body is an X-ray. In one embodiment, a representation of the body shows changes between two different representations.

As used herein, the term “assigning” refers to categorizing or labeling specific things, e.g., points, areas, or volumes of the body. In one embodiment, assigning refers to marking areas of the body with a machine learning program. In one embodiment, assigning refers to detecting landmarks. In one embodiment, assigning comprises marking changes in two representations of a body.

As used herein, the term “landmark” refers to a particular reference part of a thing. In one example, an area of the body where a bone is on the surface. Landmarks are often used as a reference point of the body to find other structures. In one embodiment, a landmark is used to measure proportion. In one embodiment, a landmark is used to find a form. In one embodiment, a landmark is a bone joint. In one embodiment, a landmark is the vertebrae. In one embodiment, a landmark is the armpit. In one embodiment, a landmark is a spine curve. In one embodiment, a landmark is a shoulder; left, right, or both. In one embodiment, a landmark is a hip; left right, or both. In one embodiment, a landmark is a knee; left, right, or both. In one embodiment, a landmark is an ankle; left, right, or both. In one embodiment, the landmarks are based on a coordinate system unique to an individual body. In one embodiment, a landmark is used as a reference point for monitoring changes of a body.

As used herein, the term “assigning landmark” refers to designating, labeling, and/or marking a particular reference part of a thing. In one embodiment, assigning a landmark comprises labeling a bone joint. In one embodiment, assigning a landmark comprises labeling the centerline of the front of the body. In one embodiment, assigning a landmark comprises labeling the centerline of the back. In one embodiment, assigning a landmark comprises labeling the shoulder; left, right, or both. In one embodiment, assigning a landmark comprises labeling the hip; left, right, or both. In one embodiment, assigning a landmark comprises labeling the knee; left, right, or both. In one embodiment, assigning a landmark comprises labeling the ankle; left, right, or both. In one embodiment, assigning a landmark is for monitoring changes around the landmark.

As used herein, the term “point within the three-dimensional representation of the body” refers to a specific position within a portrayal of a physical structure with reference to the physical structure's length, width, and height/depth. In one embodiment, the point within the three-dimensional representation of the body is a landmark. In one embodiment, the point within the three-dimensional representation of the body is a bone joint. In one embodiment, the point within the three-dimensional representation of the body is a body part. In one embodiment, the point within the three-dimensional representation of the body is the chest. In one embodiment, a point within the three-dimensional representation of the body is for monitoring changes occurring around that position.

As used herein, the term “registering” refers to matching and/or aligning two objects based on a set of features. In one embodiment, “registering” means aligning two images, such as landmarks of a representation of a body. In one example, registering means finding the warping transforming an image so that it best fits another image. In one embodiment, registering means finding a transformation mapping an image onto another image. In one embodiment, registering allows subsequent processes to recognize an image quicker and more efficiently. In one embodiment, registering comprises recording an image of a specific landmark common to multiple bodies. In one embodiment, registering comprises utilizing a cloud database platform. In one embodiment, registering comprises implementing non-rigid registration. In one embodiment, non-rigid registration is elastic deformation.

As used herein, the term “segmenting” refers to dividing, cutting, isolating, and/or segregating a thing into separate pieces or sections. Segmenting allows one to focus on an area of interest and study that area in more detail. In one embodiment, segmenting comprises dividing pieces of a body. In one embodiment, segmenting comprises dividing images of a body. In one embodiment, segmenting comprises dividing a body into a left and right half. In one embodiment, segmenting comprises dividing the body into the head, torso, left leg, and right leg. In one embodiment, segmenting comprises dividing the torso into the chest, back, abdomen, and shoulders. In one embodiment, segmenting comprises dividing the leg muscles into the upper and lower segments. In one embodiment, segmenting comprises dividing the leg muscles into left and right segments. In one embodiment, segmenting comprises dividing the leg bones into the femur and tibia. In one embodiment, segmenting comprises using atlas based segmentation of the kidneys. In one embodiment, segmenting comprises using atlas based segmentation of the liver. In one embodiment, segmenting does not separate the segments from the whole. In one embodiment, segmenting is for focusing on particular areas of change.

As used herein, the term “segment” refers to an individual section or piece of a thing. In one embodiment, the segment is attached to the thing. In one embodiment, the segment is separated from the thing. In one embodiment, a body is separated. In one embodiment, a segment of the body is an arm. In one embodiment, a segment of the body is the torso. In one embodiment, a segment of the body is separated into more segments. In one embodiment, an arm is segmented into fingers, hand, elbow, shoulder, etc. In one embodiment, a segment is tissue of the body. In one embodiment, a segment is used to find changes between two bodies.

As used herein, the term “calculating” refers to determining a value through a computation or computations. In one embodiment, calculating comprises using a machine learning program. In one embodiment, calculating comprises manual computations. In one embodiment, calculating comprises determining the volume of a segment. In one embodiment, calculating comprises determining the amount of fat in a torso. In one embodiment, calculating comprises measuring the muscle mass of an arm. In one embodiment, calculating comprises determining the volume difference between two segments. In one embodiment, calculating comprises measuring the fat difference between representations of a body.

As used herein, the term “machine learning program” refers to a type of artificial intelligence providing an apparatus, e.g., a computer, the ability to comprehend material without being explicitly instructed. In one embodiment, a machine learning program is taught to learn languages. In one embodiment, a machine learning program predicts the weather based on weather changes. In one embodiment, a machine learning program filters spam. In one embodiment, a machine learning program is taught to mark landmarks in different bodies. In one embodiment, the machine learning program is taught to detect centroids of the vertebrae (e.g., L5-T8). In one embodiment, a machine learning program automates the methods disclosed herein. In one embodiment, the number of variables determines the number of features. In one embodiment, multivariables result in either a classification or regression analysis.

As used herein, the term “volume” refers to the amount of three-dimensional space. Examples of measurements of volume include, but are not limited to, cubic inches, cubic feet, cubic centimeters, cubic meters, cubic millimeters, and cubic liters. In one embodiment, volume correlates with the distribution of weight within the body. In one embodiment, volume comprises the fat content of the body. In one embodiment, volume comprises a measurement of total body fat. In one embodiment, volume comprises a measurement of total muscle tissue. In one embodiment, volume comprises a measurement of a muscle group. In one embodiment, volume comprises a measurement of the thigh muscle. In one embodiment, volume comprises a measurement of subcutaneous fat. In one embodiment, volume comprises a measurement of the visceral fat. In one embodiment, volume comprises a measurement of the liver fat. In one embodiment, volume comprises a measurement of the intramuscular fat.

As used herein, the term “volume of the first segment” refers to the amount of space occupied by one section or piece of a thing, e.g., those corresponding with an individual piece of the body. In one embodiment, the volume of the first segment is the weight of the body, a part of the body, or parts of the body. In one embodiment, the volume of the first segment is the total fat content of the torso. Within the context of this disclosure, similar meaning is applied to “volume of the second segment”, “volume of the third segment”, “volume of the fourth segment”, and so on.

In one embodiment, the first plurality of two-dimensional images is for the same body as the second plurality of two-dimensional images. In one embodiment, the same body is the same person. In one embodiment, the same body is used for monitoring changes over time. In one embodiment, the same body changes over time.

In one embodiment, the methods disclosed herein comprise comparing the volume of the first segment to the volume of the second segment.

As used herein, the term “comparing” refers to determining the similarities and differences between two or more things. In one embodiment, comparing is between two bodies. In one embodiment, comparing is between two human bodies. In one embodiment, comparing comprises using a machine learning program. In one embodiment, comparing refers to juxtaposing two patches, such as patch A and patch B. For example, in one embodiment, comparing patch A and patch B means ordering the mean intensity of patch A and patch B. For example, patch A and patch B may be ordered where the mean intensity of patch A is greater than the mean intensity of patch B.

In one embodiment, the methods disclosed herein comprise co-registering the volume of the first segment with the volume of the second segment.

As used herein, the term “co-registering” refers to matching and/or aligning two or more objects based a set of features at the same time. In one embodiment, co-registering comprises aligning two images, such as landmark registration for the first body and the second body. In one embodiment, co-registering means finding the warping transforming an image so that it best fits another image for the first body and second body.

In one embodiment, the methods disclosed herein comprise quantifying a measure of change between the first segment and the second segment.

As used herein, the term “quantifying” refers to measuring, calculating, and/or expressing the value of an amount or measurement. In one embodiment, quantifying comprises measuring the amount of fat in a segment. In one embodiment, quantifying comprises calculating the volume of fat in a segment. In one embodiment, quantifying comprises a using computer algorithm. In one embodiment, quantifying comprises manually calculating values. In one embodiment, quantifying comprises measuring total fat. In one embodiment, quantifying comprises measuring muscle volume. In one embodiment, quantifying comprises using a machine learning program.

As used herein, the term “measure of change” refers to a magnitude of difference. In one embodiment, a measure of change is a real number. In one embodiment, a measure of change comprises units, e.g., mass, pounds, meters, etc. In one embodiment, a measure of change is used for illustrating a change over time. In one embodiment, a measure of change is the difference in fat between two bodies. In one embodiment, a measure of change is the mass lost between two time periods. In one embodiment, a measure of change is the amount of muscle mass gained over a period of time. In one embodiment, a measure of change is an average amount of fat lost during a set time period. In one embodiment, a measure of change is a vector.

In one embodiment, the methods disclosed herein comprise presenting differences between the volume of the first segment and the volume of the second segment.

As used herein, the term “presenting” refers to illustrating, displaying, showing, and/or demonstrating. In one embodiment, presenting comprises displaying three-dimensional representations of a body. In one embodiment, presenting comprises comparing three-dimensional representations of a body. In one embodiment, presenting comprises utilizing a computer algorithm. In one embodiment, presenting comprises displaying on a computer screen.

As used herein, the term “presenting difference” refers to illustrating, displaying, showing, and/or demonstrating a change. In one embodiment, presenting a difference comprises illustrating the difference in total fat volume between two bodies. In one embodiment, presenting a difference comprises showing the change in muscle mass over time. In one embodiment, presenting a difference is shown on a graphical user interface. In one embodiment, presenting differences comprises a measure of change.

In one embodiment, the methods disclosed herein comprise presenting differences in a graphical representation of the body.

As used herein, the term “graphical representation of a body” refers to a portrayal of a physical structure having material substance within a visual art medium. In one embodiment, a graphical representation of a body comprises a computer screen. In one embodiment, a graphical representation of a body comprises coordinates. In one embodiment, a graphical representation of a body comprises a graph. In one embodiment, a graphical representation of the body comprises an image. In one embodiment, a graphical representation of a body comprises volume. In one embodiment, a plurality of graphical representations of a body are used to demonstrate changes over time.

In one embodiment of the methods disclosed herein, the graphical representation of the body comprises a simulation of change in segment volume.

As used herein, the term “simulation of change” refers to a concrete representation of a difference. In one embodiment, a simulation of change comprises a presentation on a computer screen. In one embodiment, a simulation of change comprises a three-dimensional representation of a body. In one embodiment, a simulation of change comprises a difference in the total fat of a segment. In one embodiment, a simulation of change comprises a difference in the muscle structure of the same body between two points in time.

In one embodiment, the methods disclosed herein comprise a simulation of change in muscle volume.

As used herein, the term “muscle” refers to soft tissue within in a body. Muscle cells contain protein filaments, e.g., actin and myosin, sliding past one another producing a contraction that changes both the length and the shape of the cell. In one embodiment, muscles function to produce force and motion. In one embodiment, muscles are primarily responsible for maintaining and changing posture, locomotion, as well as movement of internal organs, such as the contraction of the heart, and the movement of food through the digestive system via peristalsis. In one embodiment, the muscle is skeletal/striated. In one embodiment, the muscle is cardiac. In one embodiment, the muscle is smooth.

As used herein, the term “muscle volume” refers to the amount of space occupied by soft tissue within a body. In one embodiment, muscle volume is expressed in cubic milliliters. In one embodiment, muscle volume is an embodiment in a display on a three-dimensional representation of a body. In one embodiment, muscle volume is an embodiment in a representation of the body. In one embodiment, muscle volume is a measure of change. In one embodiment, muscle volume is quantified to show a change between two bodies. In one embodiment, muscle volume is monitored over a period of time for a body.

In one embodiment, the methods disclosed herein comprise a simulation of change in fat volume.

As used herein, the term “fat” refers a biological material having both structural and metabolic functions. Fat is one of the three main macronutrients serving as an important foodstuff for many forms of life. In one embodiment, fat is a natural oily or greasy substance occurring in human bodies, especially when deposited as a layer under the skin or around certain organs. In one embodiment, fat is a triglyceride, an ester of three fatty acid chains and alcohol glycerol.

The terms “oil”, “fat”, and “lipid” are often confused and used interchangeably. “Oil” normally refers to a fat with short or unsaturated fatty acid chains and normally liquid at ambient temperatures. “Fat” may specifically refer to fats that are solid at ambient temperatures. “Lipid” is a general term, as a lipid is not necessarily a triglyceride. Fats, like other lipids, are generally hydrophobic, and are soluble in organic solvents and insoluble in water. Fats and oils are categorized according to the number and bonding of the carbon atoms in the aliphatic chain. Fats that are saturated fats have no double bonds between the carbons in the chain. Unsaturated fats have one or more double bonded carbons in the chain. Nomenclature is based on the non-acid (non-carbonyl) end of the chain, called the omega end or the n-end.

Some oils and fats have multiple double bonds and are therefore called polyunsaturated fats. Unsaturated fats can be further divided into cis fats, which are the most common in nature, and trans fats, which are rare in nature. Unsaturated fats can be altered by reaction with hydrogen affected by a catalyst. This action, called hydrogenation, tends to break all the double bonds and makes a fully saturated fat. However, trans fats are generated during hydrogenation as contaminants created by an unwanted side reaction on the catalyst during partial hydrogenation.

In one embodiment, fat is the adipose, or fatty tissue, which serves as the body's means of storing metabolic energy over extended periods of time. Adipocytes (fat cells) store fat derived from the diet and from liver metabolism. Under energy stress these cells may degrade their stored fat to supply fatty acids and also glycerol to the circulation. These metabolic activities are regulated by several hormones (e.g., insulin, glucagon and epinephrine).

As used herein, the term “fat volume” refers to the amount of biological material, having both structural and metabolic functions, occupies space in the body. In one embodiment, the fat volume is measured in cubic milliliters. In one embodiment, fat volume comprises a presentation on a graphical representation of a body. In one embodiment, fat volume is highlighted on a three-dimensional representation of a body. In one embodiment, fat volume is used for comparing two bodies. In one embodiment, fat volume is monitored over a period of a time for a body.

In one embodiment, the methods disclosed herein comprise a simulation of change in body weight.

As used herein, the term “body weight” refers to the mass of physical structure having substantial material, .e.g., an organism. In one embodiment, body weight is expressed in grams. In one embodiment, body weight is expressed in pounds. In one embodiment, body weight is the mass of a human body. In one embodiment, body weight refers to the mass of a body without any items, e.g., clothes, accessories, etc.

In one embodiment, the methods disclosed herein comprise a simulation of change in body mass index.

As used herein, the term “body mass index” or “BMI” refers to a value determined by a body's height and mass. BMI is a tool used for determining physical health by evaluating the amount of tissue mass in an individual. BMI is used for evaluating the thickness or thinness of individuals. In one embodiment, BMI is calculated by dividing the mass by the height squared, having the units kg/m2.

In one embodiment, the methods disclosed herein comprise presenting differences at three or more time intervals.

As used herein, the term “three or more time intervals” refers to greater than three different points in time. In one embodiment, the methods disclosed herein allow for the evaluation of compositional body changes over any period of time. In one embodiment, three or more intervals occur within a week. In one embodiment, three or more intervals occur within a month. In one embodiment, three or more intervals occur within a year. In one embodiment, three or more intervals occur within a decade. In one embodiment, three or more intervals occur within more than a decade.

In one embodiment, the methods disclosed herein comprise a graphical user interface for selecting one or more points in time for comparison.

As used herein, the term “graphical user interface” refers to a visual platform on a display allowing a user to control information. In one embodiment, a graphical user interface comprises a computer screen. In one embodiment, a graphical user interface comprises a three-dimensional representation of a body. In one embodiment, a graphical user interface comprises a representation of a segment of a body. In one embodiment, the graphical user interface comprises an input for a user to control the orientation of the body, e.g., using a mouse to move the body around a screen. In one embodiment, the graphical user interface comprises a zoom function so a user can control the focus of a body or body part. In one embodiment, the graphical user interface allows a user to set the parameters of a display, e.g., contouring, highlighting, marking, etc. In one embodiment, the graphical user interface comprises a plurality of displays of a plurality of bodies. In one embodiment, the graphical user interface comprises an overlap of bodies to shows changes. In one embodiment, the graphical user interface provides a timeline of changes. In one embodiment, the graphical user interface provides a pie chart. In one embodiment, the graphical user interface provides a bar graph.

As used herein, the term “selecting” refers to choosing, picking, sorting, and/or curating. In one embodiment, selecting comprises choosing a segment of a body. In one embodiment, selecting comprises choosing a body among a plurality of bodies. In one embodiment, selecting comprises choosing a time interval among a plurality of time intervals. In one embodiment, selecting comprises using a graphical user interface in which a user uses an input device, e.g., mouse, keyboard, etc., to pick segments of a body. In one embodiment, selecting comprises choosing a measure of magnitude, e.g., total fat, muscle mass, body weight, BMI index, muscle volume, fat volume, etc.

As used herein, the term “point in time” refers to a specific time, including a year, month, week, and/or day. In one embodiment, a point in time is when the first segment is created. In one embodiment, a point in time is when the second segment is created. In one embodiment, a point in time is a week after the first segment is created. In one embodiment, a point in time is a month after the first segment is created. In one embodiment, a point in time is a year after the first segment is created. In one embodiment, a point in time is a decade after the first segment is created. In one embodiment, a point in time is more than a decade after the first segment is created. In one embodiment, there is a plurality of points in time. In one embodiment, there is a plurality of points in time for a plurality of bodies. In one embodiment, a point in time is between when the first and second segment is created.

Within the context of this disclosure, the process steps or procedures can be executed by a human, operator, machine, and/or any combination thereof. For example, any combination of the following non-limiting exemplary steps could be executed by a human, operator, machine, and/or any combination thereof:

Converting DICOM to NIfTI (individual slabs)

Reassembling NIfTI slabs into full volume

Detecting/correcting cardiac artifacts

Estimating spinal cord

Estimating bone-joint landmarks

Detecting and segmenting individual vertebrae

Upsampling manual segmentations using random forests with geodesic features

Converting fat and water signal intensities to relative percentages

Estimating body mask

Computing “feature vector”

Detecting and segmenting male genitalia

Detecting and segmenting arms

Estimating boundary between the legs

Partitioning the body into anatomical regions

Segmenting the lungs and trachea

Segmenting the iliopsoas muscles

Segmenting the torso muscles (chest, back, abdomen and shoulders)

Detecting and segmenting the breasts

Segmenting the major leg muscles (upper and lower, left and right)

Segmenting the major leg bones (femur and tibia, left and right)

Segmenting pelvic bone and iliacus muscles (left and right)

Segmenting kidneys

Segmenting liver

Segmenting ribcage

Segmenting subcutaneous fat

Segmenting visceral fat

Segmenting internal thigh fat

Within the context of this disclosure, “Converting DICOM to NIfTI (individual slabs)” refers to assembling DICOM files together by series and converting into NIfTI volumes.

Within the context of this disclosure, “Reassembling NIfTI slabs into full volume” refers to merging all NIfTI series into a single whole-body volume. As well as automating and implementing fat-water swap detection/correction using only the subject's scan.

Within the context of this disclosure, “Detecting/correcting cardiac artifacts” refers to detecting motion-based cardiac artifacts and removing them to improve the quality of tissue segmentations.

Within the context of this disclosure, “Estimating spinal cord” refers to estimating a continuous curve following the contour of the spinal cord using random ferns. The location of the curve is posterior to and in between the spines of an individual vertebrae. This curve is used to exclude unwanted fat/muscle tissue when defining the abdominopelvic cavity for visceral fat segmentation.

Within the context of this disclosure, “Estimating bone-joint landmarks” refers to detecting the major bone joints (shoulders, hips, knees, ankles) using training data and machine learning techniques. The estimated locations form a coordinate system unique to each subject and allows anatomically-specific partitions.

Within the context of this disclosure, “Detecting and segmenting individual vertebrae” refers to combining training datasets in a machine-learning framework to detect the centroids of the vertebrae (L5-T8). The results are used to exclude fatty tissue from the visceral fat segmentation.

Within the context of this disclosure, “Upsampling manual segmentations using random forests with geodesic features” refers to manually generated segmented organs/tissue, obtained in a downsampled space and upsampling them to full resolution using an interpolator that is guided by the signal intensities of the data.

Within the context of this disclosure, “Converting fat and water signal intensities to relative percentages” refers to overcoming the fat and water signal intensity discrepancies caused by inhomogeneities by converting them to relative percentages. They are also used to estimate the volume estimates related to fat and non-fat tissues.

Within the context of this disclosure, “Estimating body mask” refers to producing a binary mask including only tissue associated with the subject's body and separating the subject from air, the scanner table, etc.

Within the context of this disclosure, “Computing ‘feature vector’” refers to applying Principal components analysis (PCA) to each subject's body composition above the hips. This value is used to determine which subject's in the database are most similar to a new subject for atlas-based segmentation.

Within the context of this disclosure, “Detecting and segmenting male genitalia” refers to defining a search volume in a region based on the hip landmarks and assuming the genitals are the only area where fat is not located close to the body surface. The genitals are then excluded from the fat segmentation routines.

Within the context of this disclosure, “Detecting and segmenting arms” refers to transforming the coordinate system in order to identify all voxels associated with the arms and shoulders. The arms and some of the shoulder tissue are then excluded from quantitative analysis.

Within the context of this disclosure, “Estimating boundary between the legs” refers to splitting the whole body into left and right components with particular attention to separating the legs.

Within the context of this disclosure, “Partitioning the body into anatomical regions” refers to defining four major components: head, torso, left leg and right leg.

Within the context of this disclosure, “Segmenting the lungs and trachea” refers to using the lack of an MR signal to extract the lungs and trachea.

Within the context of this disclosure, “Segmenting the iliopsoas muscles” refers to atlas-based segmentation and refinement procedures applied to the iliopsoas muscles and manually generated training data.

Within the context of this disclosure, “Segmenting the torso muscles (chest, back, abdomen, and shoulders)” refers to atlas-based segmentation and refinement procedures applied to the torso muscles and manually generating training data.

Within the context of this disclosure, “Detecting and segmenting the breasts” refers to removing the non-fat breast tissue from fat segmentations using the mask of breast tissue from the torso segmentation. The mask of breast tissue from the torso segmentation is derived from manual or atlas-based segmentation techniques. Atlas-based segmentation and refinement procedures are then applied to produce a final segmentation.

Within the context of this disclosure, “Segmenting the major leg muscles (upper and lower, left and right)” refers to generating an initial mask of the left and right legs using only the subject's data. Atlas-based segmentation and refinement are then applied to produce a final segmentation.

Within the context of this disclosure, “Segmenting the major leg bones (femur and tibia, left and right)” refers to initially segmenting the femur and tibia based on the initial leg segmentation and the detected landmarks of the bone joints. The method is based on region growing and geodesic distances. Atlas-based segmentation and refinement procedures are then applied to produce a final segmentation.

Within the context of this disclosure, “Segmenting pelvic bone and iliacus muscles (left and right)” refers to atlas-based segmentation and refinement procedures applied to the pelvic bone and iliacus muscles and manually generated training data.

Within the context of this disclosure, “Segmenting kidneys” refers to atlas-based segmentation and refinement procedures applied to the kidneys and manually generated training data.

Within the context of this disclosure, “Segmenting liver” refers to atlas-based segmentation and refinement procedures applied to the liver and manually generated training data.

Within the context of this disclosure, “Segmenting ribcage” refers to estimating the position of a thin surface containing the ribs and using a rib shape model and registration on the fat percentages.

Within the context of this disclosure, “Segmenting subcutaneous fat” refers to first defining the body cavity and then estimating all fat tissue between the body cavity and boundary of the body.

Within the context of this disclosure, “Segmenting visceral fat” refers to defining the abdominopelvic cavity and eliminating all other tissues and non-relevant organs. Then, estimating all fat tissue within the abdominopelvic cavity.

Within the context of this disclosure, “Segmenting internal thigh fat” refers to using the leg bone and subcutaneous fat segmentations for segmenting the remaining fat and muscle tissue in the upper legs. The midpoint between the hips and knees is estimated and a fixed region of muscle tissue is defined.

In one embodiment, 20 subjects are matched to a current body to provide atlases for each muscle group. The torso/iliopsoas atlases are generated by a human operator. Then, leg atlases are generated automatically. Atlas-based registration constructs a probability mask. Refinement for torso-muscle segmentation is made using graph cuts (continuous max flow). Refinement of leg-muscle segmentation are made using conditional random field (dense CRF). There is no refinement of the iliopsoas segmentation, only thresholding.

In one embodiment, data from single MRI is used. A human operator estimates the body cavity as well estimating the subcutaneous fat by excluding the body cavity. Refinement of both segmentations is done automatically by conditional random field (dense CRF). Unions and intersections of anatomy are used to estimate the abdominal cavity. Then the body cavity, pelvic mask, lungs, spine, upper legs, torso muscle, and iliopsoas muscle are estimated. Only the visceral fat is estimated in the abdominal cavity.

In one embodiment, thigh muscles are isolated. A human operator segments the subcutaneous fat from the thigh. A machine learning program automatically removes the skeletal structure. The fat fraction is calculated from a Dixon MRI. Mapping T2 and fc-T2 is from additional sequencing.

Although the disclosed invention has been described with reference to various exemplary embodiments, it is to be understood that these embodiments are merely illustrative of the principles and applications of the present invention. Those having skill in the art would recognize that various modifications to the exemplary embodiments may be made, without departing from the scope of the invention.

Moreover, it should be understood that various features and/or characteristics of differing embodiments herein may be combined with one another. It is therefore to be understood that numerous modifications may be made to the illustrative embodiments and that other arrangements may be devised without departing from the scope of the invention.

Furthermore, other embodiments of the invention will be apparent to those skilled in the art from consideration of the specification and practice of the invention disclosed herein. It is intended that the specification and examples be considered as exemplary only, with a scope and spirit being indicated by the claims.

Finally, it is noted that, as used in this specification and the appended claims, the singular forms “a,” “an,” and “the,” include plural referents unless expressly and unequivocally limited to one referent, and vice versa. As used herein, the term “include” or “comprising” and its grammatical variants are intended to be non-limiting, such that recitation of an item or items is not to the exclusion of other like items that can be substituted or added to the recited item(s).

What is claimed is:

1. A method of measuring compositional change in a body, comprising:
Collecting a first plurality of two-dimensional images for a body at a first time;
Collecting a second plurality of two-dimensional images for a body at a second time;
Processing the first plurality of two-dimensional body images into a first three-dimensional representation of the body;
Processing the second plurality of two-dimensional body images into a second three-dimensional representation of the body;
Assigning landmarks to points within the first three-dimensional representation of the body;
Assigning landmarks to points within the second three-dimensional representation of the body;
Registering the first three-dimensional representation of the body;
Registering the second three-dimensional representation of the body;
Segmenting the first three-dimensional representation of the body into a first segment;
Segmenting the second three-dimensional representation of the body into a second segment;
Calculating a volume of the first segment; and
Calculating a volume of the second segment.

2. The method of claim 1, wherein the first plurality of two-dimensional images is for the same body as the second plurality of two-dimensional images.

3. The method of claim 2, comprising:
comparing the volume of the first segment to the volume of the second segment.

4. The method of claim 2, comprising co-registering the volume of the first segment with the volume of the second segment.

5. The method of claim 2, comprising quantifying a measure of change between the first segment and the second segment.

6. The method of claim 2, comprising presenting differences between the volume of the first segment and the volume of the second segment.

7. The method of claim 6, comprising, presenting differences in a graphical representation of the body.

8. The method of claim 7, wherein the graphical representation of the body comprises a simulation of change in segment volume.

9. The method of claim 8, comprising a simulation of change in muscle volume.

10. The method of claim 8, comprising a simulation of change in fat volume.

11. The method of claim 8, comprising a simulation of change in body weight.

12. The method of claim 8, comprising a simulation of change in body mass index.

13. The method of claim 7, comprising presenting differences at three or more time intervals.

14. The method of claim 14, comprising a graphical user interface for selecting one or more points in time for comparison.
